(19) United States
(12) Patent Application Publication
(10) Pub. No.: US 2008/0146488 A1
(43) Pub. Date: Jun. 19, 2008

(54) Complexed Polypeptide and Adjuvant for Improved Vaccines

(75) Inventors: Peter J. Wettstein, Rochester, MN (US); Michael A. Strausbauch, Rochester, MN (US); Heather A. Hardin, Milton, WI (US); Nancy Borson, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH

(21) Appl. No.: 10/587,925

(22) PCT Filed: Feb. 4, 2005
(86) PCT No.: PCT/US2005/003754

§ 371 Date: Dec. 4, 2006

Related U.S. Application Data
(60) Provisional application No. 60/542,371, filed on Feb. 6, 2004.

(51) Int. Cl. A61K 38/02; A61P 35/00

(52) U.S. Cl. 514/2

(57) ABSTRACT

Attachment of the strongly immunogenic polypeptide to a more weakly immunogenic polypeptide can precipitate and focus a CpG adjuvant to increase in vivo priming of a cytotoxic T-lymphocyte (CTL) response, and thus increase the immunogenicity of the more weakly immunogenic polypeptide. Accordingly, compositions that include a bipartite immunogenic polypeptide are provided herein. The bipartite polypeptide can include a CpG-interacting amino acid sequence fused to a CTL-activating amino acid sequence that can be heterologous to the CpG-interacting amino acid sequence. Also provided are methods of identifying and using a CpG-interacting amino acid sequence and a bipartite immunogenic polypeptide.

### RELATED APPLICATIONS

This application is the National Stage of International Application No. PCT/US2005/003754, filed on Feb. 4, 2005, which claims the benefit of U.S. Provisional Application No. 60/542,371, filed Feb. 6, 2004. The contents of both applications are incorporated herein by reference in their entirety.

### GOVERNMENT SUPPORT

The work described herein was carried out, at least in part, using funds from the U.S. government under grant number AI-16052 awarded by THE NATIONAL INSTITUTES OF HEALTH. The government may therefore have certain rights in the invention.

### TECHNICAL FIELD

This invention relates generally to the field of immunotherapy, and more particularly to enhanced immunogenic polypeptides.

### BACKGROUND

Tumor-specific cytotoxic T-lymphocytes (CTLs) have been a principal focus for immunotherapy because of their exquisite specificity in lysing tumor cells with limited collateral damage. The objective of such immunotherapy is the priming for expansion of CTLs that are specific for tumor-specific polypeptides.

Proteins encoded by genes mapping to the major histocompatibility complex (MHC) present short polypeptides to cytotoxic and helper T cells. These short polypeptides provide the stimulus for activation and expansion of specific T cells. There has been a major focus on using these immunogenic polypeptides to vaccinate individuals for protection against infectious agents and tumors. The principal focus has been on polypeptides that are presented by MHC class I molecules to cytotoxic T lymphocytes (CTLs). In the case of human cancers, these polypeptides are usually injected in an oil emulsion along with cytokines and adjuvants. Importantly, the polypeptides that are used are generally of lengths that optimally bind to MHC class I molecules.

### SUMMARY

The invention is based, at least in part, on the discovery that a short, strongly immunogenic polypeptide attached to a more weakly immunogenic polypeptide can complement the CpG adjuvant to increase in vivo priming of a cytotoxic T-lymphocyte (CTL) response, and thus increase the immunogenicity of the more weakly immunogenic polypeptide. The strongly immunogenic polypeptide (referred to herein as a “CpG-interacting amino acid sequence”) can include at least one cysteine (Cys) residue and, optionally, at least one positively charged amino acid residue. Furthermore, the CpG molecule can include at least one sulfur atom, such as in a phosphorothioate diester linkage. Accordingly, the invention provides compositions, including a bipartite immunogenic polypeptide that includes a CpG-interacting amino acid sequence fused to a CTL-activating amino acid sequence that can be heterologous to the CpG-interacting amino acid sequence. Also provided are methods of identifying and using a CpG-interacting amino acid sequence and a bipartite immunogenic polypeptide, such as for enhanced immunogenicity.

In one aspect, a composition containing a polypeptide and a CpG molecule is provided. The polypeptide can include (1) an amino acid sequence that can activate a cytotoxic T lymphocyte (CTL) (referred to herein as a “CTL-activating sequence”), and (2) a CpG-interacting amino acid sequence that is heterologous to the CTL-activating sequence. The CpG-interacting amino acid sequence can include at least one cysteine residue and, optionally, at least one positively charged amino acid residue, and the CpG molecule can include at least one sulfur atom. The CpG-interacting amino acid sequence can include no more than about 15 (e.g., 12, 10, 8, or 6) amino acid residues, and the amino acid sequence can include a B-X or X-B sequence, or a B-X-B sequence, where B is a positively charged amino acid residue and X is any amino acid residue. The B residue can be, for example, an arginine or lysine. In one embodiment, the CpG-interacting amino acid sequence can be B-X-B-X-B, B-X-X-B-X-B, B-X-X-B-X-X-B, and the like. Furthermore, the amino acid sequence can include at least one cysteine residue and at least one (e.g., 2, 3, 4 or more) positively charged amino acid residues. For example, the positively charged amino acid residues can flank the Cys residue. In one embodiment, the CpG-interacting amino acid sequence includes the sequence KCSRNR (SEQ ID NO:1).

In some embodiments, the CpG-interacting polypeptide may contain a cysteine residue and no positively charged amino acids. The cysteine residue can facilitate the interaction with the CpG molecule to create a complex with enhanced immunogenicity. The CpG-interacting amino acid sequence can therefore include the sequence XCX, where C is cysteine, and X is any amino acid. For example, in one embodiment, the CpG-interacting amino acid sequence includes the sequence ACSANA (SEQ ID NO:2).

In one embodiment, the CTL-activating amino acid sequence is not longer than about 50, amino acid residues (e.g., about 25, 20, 15, or 10 amino acids, or less), and in another embodiment the entire polypeptide (CTL-activating sequence+CpG-interacting amino acid sequence) is less than 50 amino acid residues (e.g., about 40, 35, 30, 25, or 20 amino acids, or less) in length.

In one embodiment, the CpG molecule of a composition has a phosphorothioate backbone.

Also provided herein are methods for producing a composition that has enhanced immunogenicity. One exemplary method includes (a) obtaining a polypeptide that has a CTL-activating sequence and a CpG-interacting amino acid sequence, and (b) contacting the polypeptide to a CpG molecule containing a sulfur atom. The CTL-activating amino acid sequence can be heterologous to the CpG-interacting amino acid sequence, and the CpG-interacting amino acid sequence can include at least one cysteine residue and, optionally, at least one positively charged amino acid residue.

Also provided is a solution, such as an aqueous solution, that contains a precipitate. A “precipitate,” as referred to herein, is a solid material visible by the naked eye or by light microscopy. A precipitate of a solution can contain a polypeptide, and a CpG molecule. As described above, the polypeptide can include a CTL-activating sequence and a CpG-interacting amino acid sequence. The CpG-interacting amino acid sequence can include at least one cysteine residue and, optionally, at least one positively charged amino acid, and the CpG molecule can include a sulfur atom.

The methods provided herein also include a method for making a solution. One such method includes obtaining a polypeptide having a CTL-activating sequence and a CpG-interacting amino acid sequence, and contacting the polypeptide to a CpG molecule containing at least one sulfur atom. The contacting step can be performed in a solution, such as an aqueous solution, and under conditions favorable for precipitate formation between the polypeptide and the CpG molecule. The CTL-activating sequence can be heterologous to the CpG-interacting amino acid sequence, and the CpG-interacting amino acid sequence can include at least one cysteine residue and at least one positively charged amino acid residue.

Methods for activating cytotoxic T lymphocytes are also provided. For example, one method for activating cytotoxic T lymphocytes in a mammal includes administering a composition having a polypeptide and a CpG molecule to a mammal. As described above, the polypeptide can include a CTL-activating sequence and a CpG-interacting amino acid sequence that is heterologous to the CTL-activating sequence. The CpG-interacting amino acid sequence can include at least one cysteine residue and, optionally, at least one positively charged amino acid residue, and the CpG molecule can include at least one sulfur atom.

Screening methods are also provided. One such screening method includes a means of identifying a polypeptide that activates cytotoxic T lymphocytes. The method includes (a) combining a test polypeptide with a CpG molecule to form a mixture; (b) administering the mixture to a mammal, such as a mouse or a rat; (c) harvesting cytotoxic T lymphocytes from the mammal, such as from the spleen or lymph nodes of the mammal; and (d) determining whether or not the level of CD8⁺ cytotoxic T lymphocytes in the mammal is increased compared to the level of CD8⁺ cytotoxic T lymphocytes in the mammal before step (b). An increase in the level of CD8⁺ cytotoxic T lymphocytes indicates that the test polypeptide can activate cytotoxic T lymphocytes.

Another screening method can be used to identify a CpG-interacting amino acid sequence. According to one such method, a test amino acid sequence is contacted (e.g., in solution) with a CpG molecule, and it is determined whether or not the test amino acid sequence and the CpG molecule can form a precipitate. The formation of a precipitate can indicate that the test amino acid sequence is a CpG-interacting amino acid sequence. The determination step can be performed, for example, by direct visualization, such as by looking for the formation of a precipitate in the solution. The determination step can also be performed by measuring the absorbance of the solution, such as comparing absorbance of the solution before and after the contacting step. A polypeptide that will form a precipitate with a CpG molecule is a candidate polypeptide that may be further tested for an ability to activate cytotoxic T lymphocytes. However, a polypeptide that does not precipitate a CpG molecule is not necessarily incapable of activating cytotoxic T lymphocytes, particularly if the polypeptide includes a cysteine.

Also provided are methods for identifying a candidate CpG-interacting amino acid sequence. In one embodiment, the method includes (a) administering a polypeptide/CpG molecule mixture to a mammal, and (b) determining whether or not the mixture activates CTLs from the mammal to a level greater than the level of activation that occurs in a control mammal that received a control polypeptide/CpG molecule mixture. The polypeptide of the polypeptide/CpG molecule mixture can include a CTL-activating amino acid sequence and a test amino acid sequence; the polypeptide of the control polypeptide/CpG molecule mixture will lack the test amino acid sequence. Determining that the level of CTL activation is greater in the presence of the test amino acid sequence is an indication that the sequence may be a CpG-interacting amino acid sequence. The determining step can include an immunocytochemical technique, such as an ELISA or ELISPOT assay.

One feature of the invention is a composition comprising a CpG molecule and a polypeptide containing more than about four cysteines (and more than about two disulfide bonds) within a sequence of about 100 contiguous amino acids. The polypeptide can be an antigenic polypeptide, such as a synthetic or a natural antigenic polypeptide. As used herein, a “natural polypeptide” contains an amino acid sequence that is found in vivo, such as in a mammal (e.g., a human).

There are many advantages to delivering a polypeptide (e.g., a polypeptide having a CpG-interacting amino acid sequence and a CTL-activating sequence) and CpG molecules together in the form of a therapeutic regimen or vaccine. Delivery of the two molecules in one immunotherapeutic composition simplifies vaccination, and in addition, can provide each component protection from degradation and facilitate transport of the components into the cell. The relative ease of producing the vaccines described herein can reduce the time required to develop a vaccine to months or weeks or even days. The relative ease can also facilitate the custom design of vaccines for individual tumor types, or particular pathogenic diseases, for timely application.

Sequestering the CpG molecule in a complex can also prevent the development of toxic, systemic responses that are frequently observed when free synthetic CpG molecules are administered. Without wishing to be bound by theory, the complexes may provide for a “timed-release” of smaller units composed of antigen and CpG.

In another advantage, the complexing phenomenon can be used for targeting small molecules for intracellular delivery, such as for gene therapy applications.

The development of effective, polypeptide-based vaccines for a number of different types of human cancers has been pursued in order to take advantage of the selective expression of specific polypeptides in those tumors. These efforts have principally focused on the use of polypeptides with lengths that are optimal for direct presentation to effector T lymphocytes. The compositions described herein, including the vaccine polypeptides described herein can be optimized specifically for in vivo priming through consideration of the mechanisms that regulate presentation of polypeptides by professional antigen presenting cells (APCs) to effector T cells.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, useful methods and materials are described below. The materials, methods, and examples are illustrative only and not intended to be limiting. Other features and advantages of the invention will be apparent from the accompanying drawings and description, and from the claims. The contents of all references, pending patent applications and published patents, cited throughout this application are hereby expressly incorporated by reference. In case of conflict, the present specification, including definitions, will control.

### DESCRIPTION OF DRAWINGS

FIG. 1A is a graphical presentation of the frequencies of IFNγ-secreting CTLs specific for three minor H antigen peptides, H60 (SEQ ID NO:19), HY1 (SEQ ID NO:9), and HY2 (SEQ ID NO:10). Stimulators included syngeneic and allogeneic spleen cells.

FIG. 1B is a graphical representation of the frequencies of IFNγ-secreting CTLs specific for three minor H antigen polypeptides, H60 (SEQ ID NO:19), HY1 (SEQ ID NO:9), and HY2 (SEQ ID NO:10). Stimulators included RMA/S cells pulsed with the respective target peptides at concentrations of 10 nM.

FIG. 2 is a graphical presentation of the frequencies of IFNγ-secreting CTLs in response to different concentrations of the indicated antigen polypeptides (HY2, HY1, KCSRNR-HY1 (SEQ ID NO:20), and RKKRRQ-HY1 (SEQ ID NO:21)) mixed with CpG.

FIG. 3A is a graphical presentation of the frequencies of IFNγ-secreting CTLs. Mice were primed with CpG mixed with HY1 and the indicated HY1 polypeptides (SEQ ID NOs 20, 22, 23, 24 and 18, respectively). CD8⁺ CTLs were mixed in a primary IFNγ Elispot assay with syngeneic male and female spleen cell stimulators.

FIG. 3B is a graphical presentation of the frequencies of IFNγ-secreting CTLs. B6 female mice were primed with CpG mixed with HY1 and the indicated HY1 polypeptides (SEQ ID NOs 20, 22, 23, 24 and 18, respectively). A polypeptide that included a Cys-containing amino and carboxy terminal tail was included. CD8⁺ CTLs were mixed in a primary IFNγ Elispot assay with B6 female spleen cells that were untreated or pulsed with 1 μM HY1 peptide.

FIG. 4 is a graphical presentation of the frequencies of IFNγ-secreting CTLs, when the spleen cells from the B6 female responders described in FIG. 3B were re-stimulated with B6 male stimulator cells in primary MLCs. CD8⁺ CTLs were enriched from the surviving cells and mixed in a secondary IFNγ Elispot assay with B6 male spleen cells, untreated B6 female spleen cells, and B6 female spleen cells pulsed with 1 μM HY1 peptide.

FIG. 5A is a graphical presentation of the frequencies of IFNγ-secreting CTLs detected in primary IFNγ Elispot assays. B6 female mice were primed with CpG+peptide in titrated doses (100, 40, 10 μg of each component) and spleen cells were harvested after 10 d for enrichment of CD8+ CTLs. CTLs were mixed in primary IFNγ Elispot assays with B6 male and female stimulators

FIG. 5B is a graphical presentation of the frequencies of IFNγ-secreting CTLs detected in primary IFNγ Elispot assays. B6 female mice were primed with CpG+peptide in titrated doses (100, 40, 10 μg of each component) and spleen cells were harvested after 10 d for enrichment of CD8+ CTLs. CTLs were mixed in primary IFNγ Elispot assays with B6 female stimulators that were untreated and pulsed with 10 nM HY1 peptide.

FIG. 6A is a graphical presentation of the frequencies of IFNγ-secreting CTLs detected in primary IFNγ Elispot assays. B6 female mice were primed with CpG plus KCSRNR-HY1 (SEQ ID NO:20) and ACSANA-HY1 (SEQ ID NO:23) and spleens and draining lymph nodes were harvested at 15, 29, and 50 days. CD8⁺ CTLs were enriched and mixed in primary IFNγ Elispot assays with untreated B6 female spleen cells or B6 female cells pulsed with 1 nM peptide. Filled and open bars correspond to draining lymph nodes and spleens, respectively. Frequencies of CTLs responding to untreated B6 female stimulators have been subtracted to yield the reported specific frequencies.

FIG. 6B is a graphical presentation of the frequencies of IFNγ-secreting CTLs detected in primary IFNγ Elispot assays. B6 female mice were primed with CpG plus KCSRNR-HY1 (SEQ ID NO:20) and ACSANA-HY1 (SEQ ID NO:23) and spleens and draining lymph nodes were harvested at 15, 29, and 50 days. CD8⁺ CTLs were enriched and mixed in primary IFNγ Elispot assays with untreated B6 female spleen cells or B6 female cells pulsed with 1 uM HY1 peptide. Filled and open bars correspond to draining lymph nodes and spleens, respectively. Frequencies of CTLs responding to untreated B6 female stimulators have been subtracted to yield the reported specific frequencies.

FIG. 7 is an example of primary ELISPOT assay results. The elimination of MPL-AF adjuvant from the priming combination of KCSRNR-HY1 (SEQ ID NO:20) and CpG appeared to increase the efficiency of priming. The wells shown include 2.5×10⁵ CD8⁺ CTLs as responders and RMA-S stimulators pulsed with HY1 polypeptide. The graph indicates frequency of activated CD8⁺ T cells in response to polypeptide stimulation.

FIG. 8 is an example of primary ELISPOT assay results. The linkage of KCSRNR (SEQ ID NO:1) CpG-interacting amino acid sequences to two melanoma CTL-activating polypeptides (AAGIGILTV (SEQ ID NO:4) (MelanA) and KTWQYWQV (SEQ ID NO:5) (gp100)) and the immunodominant influenza polypeptide (GILGFVFT (SEQ ID NO:6)) resulted in increased priming of HLA-A2 transgenics when mixed with CpG+MPL-AF in comparison to their respective native polypeptides. The wells shown include 3×10⁵ CD8⁺ CTLs as responders with T2 stimulators pulsed with polypeptide. The graph indicates frequency of activated CD8⁺ T cells in response to polypeptide stimulation.

FIG. 9 is a graphical representation of absorption data. Formation of a precipitate results in decreased absorbance. The data indicate that positively charged residues in the CpG-interacting amino acid sequences of HY1 mediate virtually complete precipitation of CpG with concentrations used for in vivo priming.

FIG. 10 is a graphical representation of absorbance values for supernatants following the precipitation of CpG from solution with HY1-tailed bipartite polypeptides. Precipitates were cleared from the supernatants, which were then diluted 1/100 for spectrometric analysis at 260 nm.

FIG. 11A is an RP-HPLC trace of a mixture of (SEQ ID NO:24) AASANA-HY1+S1-CpG, where S1-CpG contains a single phosphorothioate group. Mixtures were separated by RP-HPLC with a gradient of 0-95% acetonitrile in 0.1% trifluoroacetic acid over 75 min.

FIG. 11B is an RP-HPLC trace of a mixture of (SEQ ID NO:23) ACSANA-HY1+native CpG. Mixtures were separated by RP-HPLC as described in FIG. 10A.

FIG. 11C is an RP-HPLC trace of a mixture of ACSANA-HY1+S1-CpG (see FIG. 10A). Mixtures were separated by RP-HPLC as described in FIG. 10A.

FIG. 11D is an RP-HPLC trace of a mixture of ACSANA-HY1 (SEQ ID NO:23) and S1-CpG (see FIG. 10A), where the mixture was reduced with 0.05M DTT immediately prior to separation by RP-HPLC. Mixtures were separated by RP-HPLC as described in FIG. 10A.

FIG. 11E is an RP-HPLC trace of a mixture of ACSANA-HY1 (SEQ ID NO:23) and S1-CpG (see FIG. 10A) that had been alkylated with 10 mM iodoacetamide prior to mixing. Mixtures were separated by RP-HPLC as described in FIG. 10A.

FIG. 11F is an RP-HPLC trace of a mixture of ACSANA-HY1 (SEQ ID NO:23) and S1-CpG (see FIG. 10A). Mixtures were separated by RP-HPLC as described in FIG. 10A.

FIG. 12 is a graphical presentation of the percentage of doubly-stained Langerhans cells. B6 female mice (3/group) were anesthetized and injected in the footpads with Texas Red-conjugated CpG plus the HY1, KCSRNR-HY1 (SEQ ID NO:20), and ACSANA-HY1 (SEQ ID NO:23) peptides that had been conjugated with Alexa 488. The recipients were sacrificed after 24 hr, and the popliteal lymph nodes were dissociated. The frequencies of doubly stained cells were estimated by fluorescence microscopy.

### DETAILED DESCRIPTION

The invention is based, at least in part, on the discovery that a short, strongly immunogenic polypeptide attached to a weakly immunogenic polypeptide (referred to herein as a “CTL-activating” polypeptide) can complement a CpG adjuvant to increase the in vivo priming of a cytotoxic T-lymphocyte (CTL) response. The strongly immunogenic polypeptide (referred to herein as a CpG-interacting polypeptide) includes at least one cysteine (Cys) residue and, optionally, at least one positively charged amino acid residue. In addition, the CpG molecule includes at least one sulfur atom. Fusion of the strongly immunogenic CpG-interacting polypeptide with a CTL-activating polypeptide can produce a bipartite polypeptide featured in the invention. The amino acid sequence of the CTL-activating polypeptide can be heterologous to the amino acid sequence of the CpG-interacting polypeptide. The resulting bipartite immunogenic polypeptide can also be called a “primotope.”

The CTL-activating amino acid sequence of the bipartite immunogenic polypeptide can be a polypeptide that binds to MHC Class I molecules. Thus, the bipartite immunogenic polypeptide of the invention can also be known as a CTL-activating polypeptide with enhanced priming potential, or an MHC Class I binding polypeptide with enhanced priming potential. The enhanced priming potential comes from a short CpG-interacting amino acid sequence fused to the polypeptide that binds MHC class I molecules. The short amino acid sequence includes at least one Cys residue and, optionally, at least one (e.g., 2, 3, or more) positively charged amino acids. At least one positively charged amino acid of the CpG-interacting amino acid sequence can be Arg, Lys, or His. The total length of the CpG-interacting amino acid sequence can be less than about 20 amino acids long (e.g., less than about 15, 12, 10, 8, or 6 amino acids long).

A CpG-interacting amino acid sequence can be added to an antigen of interest in order to increase the immunogenicity of that antigen. The cysteine residues of the CpG-interacting polypeptide (and hence the antigen to which it is attached) can interact, e.g., covalently bond, with a CpG molecule. The CpG molecule is an immune system stimulant that elicits a strong cell-mediated immune response. These CpG molecules are routinely used as adjuvants in vaccine research, during which they are given in their free form (they are not routinely linked to any other molecules as described in this disclosure). However, CpG molecules are not currently approved for human use because their systemic administration triggers a toxic shock response (this is one problem that the current disclosure overcomes). The majority of CpG molecules used as adjuvants are synthesized using a phosphorothioate backbone in order to make the oligonucleotides more stable and less sensitive to nucleases. The presence of the sulfur groups in the phosphorothioate backbone of the CpG oligonucleotides may allow for the formation of disulfide bonds with the sulfur residue of the cysteines contained in the CpG-interacting amino acid sequence. This disulfide covalent linkage between the CpG-interacting amino acid sequence and the CpG oligonucleotide may help facilitate the increased antigen immunogenicity.

The aggregation of several polypeptides-CpG compounds can form a precipitate and demonstrate increased antigen immunogenicity. The positively charged amino acids of the CpG-interacting amino acid sequence can interact with the negatively charged backbone of the CpG oligonucleotides to form the aggregates. The resulting precipitate can perform two functions: (1) it can increase aggregate (antigen) uptake by antigen presenting cells (the first step in forming an immune response to the antigen) and (2) it can localize the CpG molecules to prevent systemic circulation (and hence toxic shock). The CpG-interacting amino acid sequences can have a periodicity of positively charged amino acids. Because of the helical nature of one or more CpG molecules, the negative charges are oriented in a certain way. In order for the CpG-interacting amino acid sequence to orient the positively charged amino acids into a conformation best able to bind the negative charges of the CpG molecule, single or multiple spacer amino acids can be used. A general orientation can be B-X, or X-B, or B-X-B, where B is a positively charged amino acid residue, and X is an amino acid residue. The B residue can be, for example, an arginine or lysine or histidine. In one embodiment, the CpG-interacting amino acid sequence can be B-X-B-X-B, B-X-X-B-X-B, B-X-X-B-X-X-B, and the like.

In some cases, a CpG-interacting polypeptide may contain a cysteine residue and no positively charged amino acids. The cysteine residue can facilitate the interaction with the CpG molecule to create a complex with enhanced immunogenicity. The CpG-interacting amino acid sequence can therefore have the include the sequence XCX, where C is cysteine, and X is any amino acid.

The development of immunotherapeutic methods can provide alternative treatments for human tumors that resist standard treatment methods including chemotherapy and radiation therapy. Tumor-specific cytotoxic T lymphocytes (CTLs) have been a principal focus for immunotherapy because of their exquisite specificity in lysing tumor cells with limited collateral damage. The objective of such immunotherapy is the priming for expansion of CTLs that are specific for tumor-specific polypeptides presented by products of class I genes of the major histocompatibility complex.

Class I-binding polypeptides that include multiple positively charged amino acids and at least one cysteine residue exhibit the ability to complex with and precipitate CpG molecules, and the addition of these amino acids to weakly immunogenic polypeptides increases immunogenicity when combined with CpG molecules for immunization. This increased immunogenicity is accompanied by a decrease in the systemic effects of CpG molecules suggesting that co-precipitated polypeptide and CpG molecules provide the basis for development of vaccines with increased immunogenicity and half-life with reduced adjuvant-mediated toxicity. The simultaneous delivery of two components simplifies the administration of vaccine and expectedly provides protection from degradation for each component. The relative ease of production of these vaccines would enable custom designing of vaccines for individual tumor types for timely administration.

Bipartite Immunogenic Polypeptides The bipartite immunogenic polypeptides of the invention can consist of a CpG-interacting amino acid sequence (e.g., a CpG-interacting amino acid “tail”) and a CTL-activating sequence, which can be heterologous to the CpG-interacting amino acid sequence. The CpG-interacting amino acid sequence can be located at any position in the bipartite immunogenic polypeptide, such as at, or near, the N- or C-terminus, or in about the middle of the polypeptide. The CTL-activating sequence can be an MHC Class I binding polypeptide. An immunogenic polypeptide of the invention is the shortest polypeptide that can be efficiently processed and presented by APCs, bound by class I molecules, and recognized by specific CTLs in vivo. The total length of the bipartite immunogenic polypeptide can be less than about 100 amino acids long, preferably less than about 50 amino acids long (e.g., less than about 40, 35, 30, 25, 20, or 15 amino acids long).

Fully processed polypeptides are those that optimally bind to MHC molecules for recognition by effector T cells. Without wishing to be bound by theory, the positively charged CpG-interacting amino acid sequence can provide the necessary characteristics that allow the immunogenic polypeptide of the invention to be taken up by APCs.

CpG-Interacting Amino Acid Sequence The CpG-interacting amino acid sequence can complex with and concentrate the adjuvant activity of CpG motifs for activation of localized APCs.

The CpG-interacting amino acid sequence can include 0, 1, 2, 3, or more positive amino acids. An excess of positively charged residues may block T cell activation and expansion in a short term scenario, but may be effective in stimulating T cell activation over a long term period (e.g., longer than 30, 40, 50, 75, 100, or 150 days, or longer). This inhibition may be caused by a concentrated precipitation of CpG that sequesters both the polypeptide and CpG in an inactive complex. The Cys and positively charged amino acids can occur at any position within the CpG-interacting amino acid sequence, and the other amino acids can be any amino acid, but preferably positively charged amino acids are spaced at regular intervals throughout the sequence. For example, a positively charged amino acid can be positioned at every other, every third, or every fourth amino acid position. It is not necessary that the placement of the positive amino acid residues has perfect periodicity. The CpG-interacting amino acid sequence can be, for example, any CpG-interacting amino acid sequence described herein. The CpG-interacting amino acid sequence can be, for example, KCSRNR (SEQ ID NO:1) or ACSANA (SEQ ID NO:2).

While not wishing to be bound by theory, the cysteine residues of the CpG-interacting amino acid sequence can interact with, e.g., covalently link, the CpG-interacting amino acid sequence (and so also the CTL-activating amino acid sequence to which it is attached) to a CpG molecule.

CTL-Activating Amino Acid Sequence A “CTL-activating” polypeptide is defined by a CTL-activating amino acid sequence. A polypeptide can be categorized as a CTL-activating polypeptide if administration to a mammal, such as a mouse, or a human, results in increased levels of activated CTLs. The level of activated CTLs can be monitored by a variety of methods known in the art, including, but not limited to, ELISA and ELISPOT assays.

Polypeptides that are rich in disulfide bonds (such as IgG) may not require a heterologous CpG-interacting sequence, but instead can themselves bind enough CpG molecules to elicit a heightened immune response. These highly antigenic polypeptides can have at least four cysteine residues (e.g., 5, 10, 15, 20, 25, or 30 cysteines) per 100 contiguous amino acids. The polypeptide can also be rich in disulfide bonds. For example, the polypeptide can contain at least two disulfide bonds (e.g., 3, 4, 5, 6, 7, 8, 9, 10, or 15 disulfide bonds) when it is folded in its natural state. The antigenic polypeptide can be treated with a denaturant, such as urea, guanidine chloride, guanidine thiocyanate GdmSCN, or heat and beta-mercaptoethanol, to break the disulfide bonds, and then the denatured polypeptide can be mixed with CpG molecules and a precipitate allowed to form. The resulting mixture can be used as an immunotherapeutic formulation as described herein.

CpG molecule A CpG molecule, as described herein, is an oligonucleotide that contains at least one unmethylated cytosine-guanine dinucleotide. CpG molecules can be about 15-25 nucleotides long, preferably about 18-20 nucleotides long. The oligonucleotide can include at least one CpG consensus motif of RRCpGYY (SEQ ID NO: 8) (R is purine and Y is pyrimidine). The CpG molecules can include a backbone of at least one phosphorothioate linkage, and preferably, the backbone of the entire CpG molecule consists of phosphorothioate linkages. The side chains of a phosphorothioate backbone contain at least one or more sulfur atoms in place of oxygen, and a phosphorothioate backbone can yield a longer half life, increased level of activity, and subtle changes in the specificity of activity (Kreig, Annu. Rev. Immunol. 20:709, 2002) compared to an oligonucleotide backbone that does not include a sulfur atom. While not being bound by theory, the sulfur atoms of the phosphorothioate backbone can form disulfide linkages with the Cys residue(s) of the CpG-interacting amino acid sequence, which can enhance the immunogenic response.

Any CpG molecule, or any DNA molecule with a phosphorothioate linkage for coupling through disulfide bonds, can be used in the compositions and methods described herein. For example, the CpG molecule 1826 (5′-TCC A TG ACG TTC CTG ACG TT-3′ (SEQ ID NO:16)), specific for mouse TLR (Davis et al. J. Immunol. 160:870, 1998) can be used as described. See also CpG molecules described in Lingnau et al. (Vaccine 20:3498-3508, 2002), for example.

Immunotherapeutic Formulations The compositions and methods described herein can be used in the form of vaccinations, to treat or prevent a disease or disorder, such as cancer. An exemplary immunotherapeutic composition contains a bipartite immunogenic polypeptide and CpG molecules in an oil emulsion, such as Incomplete Freund's Adjuvant. Optionally, an immunotherapeutic composition can include MPL-AF (monophosphoryl Lipid A adjuvant (MPL) mixed with dipalmitoyl phosphatidyl choline (DPPC)).

An immunotherapeutic composition described herein can contain a heterologous mixture of bipartite immunogenic polypeptides. For example, the mixture can contain polypeptides with distinct CTL-activating and CpG-interacting amino acid sequences, or one species of CTL-activating sequences can be coupled to a variety of different CpG-interacting amino acid sequences. The use of various CpG-interacting amino acid sequences can provide a collection of bipartite immunogenic polypeptides with varying stability and varying CTL activation capabilities. For example, and without wishing to be bound by theory, shorter CpG-interacting amino acid sequences containing fewer or one cysteine residue can activate CTLs in the shorter term, providing an immediate priming effect, while longer CpG-interacting amino acid sequences with multiple cysteine residues, or greater numbers of positively charged residues, can provide longer-term priming activity.

As used herein, a vaccine is a composition that provides protection against a viral infection, cancer or other disorder or treatment for a viral infection, cancer or other disorder. Protection against a viral infection, cancer or other disorder will either completely prevent infection or the tumor or other disorder, or will reduce the severity or duration of infection, tumor or other disorder if subsequently infected or afflicted with the disorder. Treatment will cause an amelioration in one or more symptoms or a decrease in severity or duration. For purposes herein, a vaccine results from administration of the bipartite immunogenic polypeptides and CpG molecules described herein. As used herein, amelioration of the symptoms of a particular disorder by administration of a particular composition refers to any lessening, whether permanent or temporary, lasting or transient that can be attributed to or associated with administration of the composition.

Administration of the immunotherapeutic compositions described herein can activate Langerhans cells and antigen presenting cells (APCs), each of which is capable of presenting the antigenic polypeptide to CTLs, thereby activating the CTLs. The immunotherapeutic compositions can also be administered with a second therapeutic agent or regimen. For example, an immunotherapeutic agent can be administered to a patient who also receives chemotherapy or radiation therapy, such as for a cancer.

An immunotherapeutic composition described herein can be provided in solution, such as in sterile water or a buffer, or the composition can be packaged in a lyophilized form. Kits containing the compositions can include solubilizing reagents, such as sterile water or buffers and/or reagents for diluting a solution and/or otherwise adjusting the properties of a solution in preparation for an intended use. A kit can also include informational material; informational material can be descriptive, instructional, marketing or other material that relates to the methods described herein and/or the use of the immunotherapeutic compositions for the methods described herein.

The informational material of the kits is not limited in its form. In many cases, the informational material is provided in printed matter, such as in printed text, drawings, and/or photographs. The instructional material can be in the form of a label or printed sheet. However, the informational material can also be provided in other formats, such as Braille, computer readable material, video recording, or audio recording. The informational material can include contact information, such as a physical address, email address, website, or telephone number, where a user of the kit can obtain substantive information about an immunotherapeutic composition and/or its use in the methods described herein. Of course, the informational material can also be provided in any combination of formats.

The compositions can be packaged in a variety of suitable containers. For example, a composition can be contained in a bottle, vial, or syringe, composed of a material such as glass or plastic. Optionally, the compositions can be packaged in individual dosage form, such as in ampoules, syringes, or blister packs. Containers can be air tight and/or waterproof, and can be labeled for use, such as for a vaccine, or to stimulate a CTL response, or to treat a cancer.

Effective Dose The compositions described herein can be administered on multiple occasions and at varying concentrations.

Toxicity and therapeutic efficacy of the compositions disclosed herein (e.g., immunotherapeutic compositions) can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from the cell culture assays and further animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any composition used in the methods described herein, the therapeutically effective dose can be estimated initially from cell culture assays. A dose can be formulated in animal models to achieve a circulating plasma concentration range that includes the IC₅₀ (that is, the concentration of the immunogenic polypeptides and CpG molecules (free and complexed) which achieves a half-maximal inhibition of symptoms, e.g., treatment of a tumor and/or CTL-activation) as determined in cell culture. Such information can be used to more accurately determine useful doses in humans. The amount of bipartite immunogenic polypeptide and CpG molecule in a vaccine dose is selected as an amount that induces an immunoprotective response without significant, adverse side effects in a vaccinee. Such amount can vary depending on the target (e.g., a tumor or systemic vaccination procedure). Generally it is expected that each dose will comprise less than about 500 μg (e.g., less than about 400, 300, 200, 100, 90, 80, 70, 60, 50, 40, 20 10, 5 or 1 μg) each of total bipartite immunogenic polypeptide and CpG molecule. The dose can, optionally, comprise an equal molar ratio of the two components. An optimal amount for a particular vaccine can be ascertained by standard studies involving observation of CTL responses, antibody titres, and other responses in subjects.

Following an initial vaccination, subjects may receive a boost in about 4 weeks. The formulations and routes of administration can be tailored to the specific disorder being treated, and for the specific human being treated. For example, the human can have a cancer, such as a leukemia, or a tumor, such as a tumor of the breast, colon, prostate, pancreas or lung.

Generally, administration of an immunotherapeutic agent facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In: Remington's Pharmaceutical Sciences, 18th Ed., Gennaro, ed., Mack Publishing Co., Easton, Pa., 1990). Generally, the dosage required to provide an effective amount of a formulation will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's The Pharmacological Basis of Therapeutics, 9th Ed., Hardman et al., eds., McGraw-Hill, New York, N.Y., 1996).

Screening Methods Various screening methods are also provided herein. One such screening method can be used to identify polypeptides that activate cytotoxic T lymphocytes. The CTL-activating polypeptides can be used in the production of the bipartite immunogenic agents described herein. The methods can include, for example, combining a test polypeptide with a CpG molecule, administering the mixture to a mammal, such as a mouse or a rat, harvesting cytotoxic T lymphocytes from the mammal, and determining whether or not the level of cytotoxic T lymphocytes (e.g., CD8⁺ CTLs) in the mammal is increased.

Other screening methods include procedures to identify a CpG-interacting amino acid sequence, such as for use in a bipartite immunogenic polypeptide. For example, a test amino acid sequence can be contacted with a CpG molecule, and the mixture observed for the formation of a precipitate. The formation of a precipitate indicates that the test amino acid sequence is a CpG-interacting amino acid sequence. The method can further include administering a bipartite immunogenic polypeptide that contains the identified CpG-interacting amino acid sequence. The polypeptide can be administered to a mammal in a formulation with a CpG molecule. By determining whether the polypeptide/CpG molecule composition can activate CTLs in the mammal, it can be determined that the test CpG-interacting amino acid sequence can function in vivo as part of an effective immunotherapeutic reagent.

Sequences identified as CTL-activating sequences or CpG-interacting amino acid sequences can be recorded in a print or machine-readable form. Further, polypeptides containing the identified sequences can be further tested in humans and assayed for an immunogenic response.

The invention is further illustrated by the following examples, which should not be construed as further limiting.

### EXAMPLES

### Example 1

An immunogenic polypeptide precipitates CpG and enhances activation of CTLs Early experiments in our lab focused on methods required to increase the T cell response to minor histocompatibility antigen (MiHA) polypeptides in mice. Over the past ten years, we have observed a continued decline in the ability of mice to generate CTLs specific for MiHA polypeptides. This decline may be due to the increasingly stringent husbandry procedures in our mouse rooms that have reduced the exposures to pathogens to the point where the immune systems of our mice have no reason to do anything more than maintain homeostasis. Since shifting to primary ELISPOT for quantitation of CTL that produce IFNgamma upon stimulation, we have observed that background spots are virtually non-existent in our mice. This is in direct contrast to primary ELISPOT assays with human CTLs, where background activity is ever-present. These observations are consistent with overall decreases in immune activity that must be overcome for effective generation of CTL responses.

Since the defect appeared to be founded in limited exposure to pathogens, we explored the use of bacterial adjuvants to increase CTL responses to MiHA rather than the use of pharmacological methods, e.g., antibody-mediated co-stimulation. We settled on the use of LipidA and a CpG molecule, that bind to TLR-4 and TLR-9, respectively, based on their reported abilities to enhance CTL responses. Lipid A was provided by Corixa Corporation (Seattle, Wash.) in the form of MPL-AF (MPL adjuvant (monophosphoryl Lipid A) mixed with surfactant-like dipalmitoyl phosphatidyl choline (DPPC)). The CpG molecule used was the 1826 oligonucleotide (TCCA TGAC GTTC CTGA CGTT (SEQ ID NO:16)) that is specific for mouse TLR-9 (Davis et al., J. Immunol. 160:870, 1998.); the CpG oligonucleotides were synthesized by the Mayo Clinic Molecular Biology Core with a phosphorothioate backbone (unless otherwise specified, our use of the term CpG will refer to this synthetic form). In an effort to avoid the intense, systemic inflammation previously observed in mice treated repeatedly with CpG, we mixed CpG, MPL, and antigen for use in single challenges. Preliminary experiments were performed with 10 μg MPL-AF+100 μg CpG mixed with either MiHA-incompatible spleen cells or synthetic MiHA polypeptides (100 μg) for sub cu (s.c.) injection in the base of the tail.

Frequencies of IFNgamma-secreting, MiHA-specific CTLs were estimated by the use of primary ELISPOT assays that utilized CD8⁺ responders that were enriched by negative selection of other lymphoid populations with MACS CD8⁺ Negative Selection Kits (Miltenyi Biotec, Auburn, Calif.).

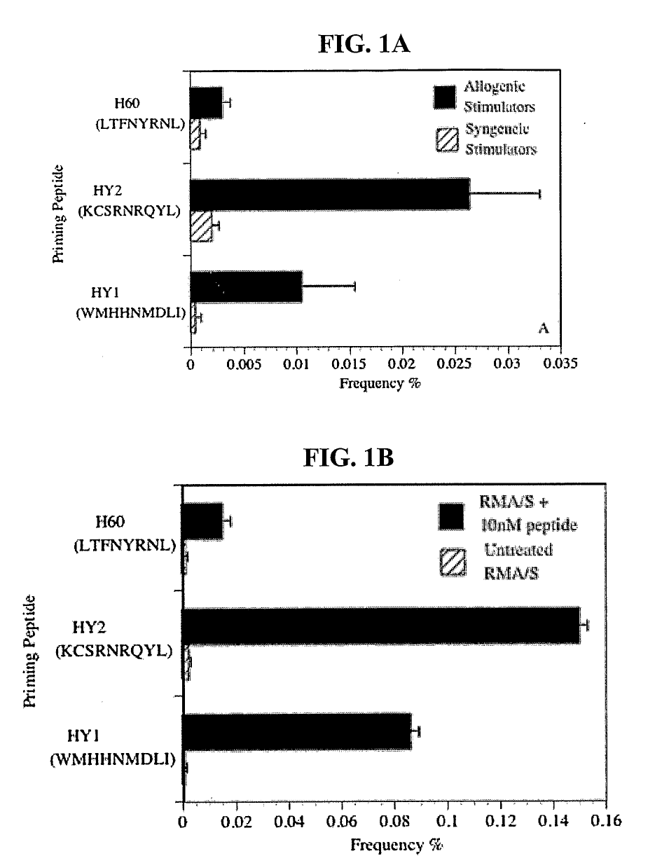

The WMHHNMDLI (HY1) (SEQ ID NO:9) and KCSRNRQYL (HY2) (SEQ ID NO:10) peptides are derived from proteins encoded by murine Y-linked genes and are presented by H2D^{b} molecules to CTLs (King et al., Genomics 24:159-168, 1994; Greenfield et al., Nat. Genetics 14:474-478, 1996). Mixtures of these two peptides and combined MPL-AF plus CpG adjuvants were tested for their capacities to prime CTL responses. These two peptides were also compared for in vivo priming efficiency with an immunodominant H60 peptide (LTFNYRNL (SEQ ID NO:19)) (Malarkannan et al., J. Immunol. 161:3501-3509, 1998). B6 females were primed s.c. and spleens were harvested 10 days after immunization. CD8⁺ CTLs were enriched by negative selection and stimulated with (1) syngeneic female spleen cells, (2) syngeneic male spleen cells, (3) allogeneic (BALB.B) spleen cells for anti-H60 CTLs, and (4) peptide-pulsed RMA/S cells in primary Elispot assays to estimate the frequencies of IFNγ-secreting CTLs. Primary Elispot assays were performed with CD8+ splenocyte responders from recipients of single immunizations with 100 μg polypeptide plus 100 μg CpG and 10 μg MPL-AF. RMA/S cells were pulsed with the respective target polypeptides at concentrations of 10 nM. Responders and stimulators were cultured in anti-IFNgamma capture antibody-coated ELISPOT plates for 48 hr after which biotinylated anti-IFNgamma detection antibodies were added followed by streptavidin-conjugated HRP and AEC substrate. Spots were counted by first obtaining digitized images of the wells (performed by C.T.L. Analyzers, Cleveland, Ohio) and then analyzing these images with Immunospot software purchased from C.T.L. Analyzers. On the basis of frequencies of responding CTLs, the HY2 polypeptide was the most efficient primer of CTLs whereas the H60 polypeptide was the most inefficient (FIGS. 1A and 1B); see also U.S. Provisional Application No. 60/542,371.

The HY2 polypeptide was distinguished from the other test polypeptides (HY1, H60, and several others) by the formation of precipitates when MPL+CpG were mixed with HY2. The precipitations only required HY2 and CpG (MPL was not required). The HY2 amino acid sequence was examined for characteristics that could promote increased immunogenicity and complexing with the CpG molecule. We tested a panel of polypeptides, some of which did not precipitate upon the addition of CpG, and found that if positively charged amino acids were added to these non-precipitating polypeptides, they now formed precipitates with CpG.

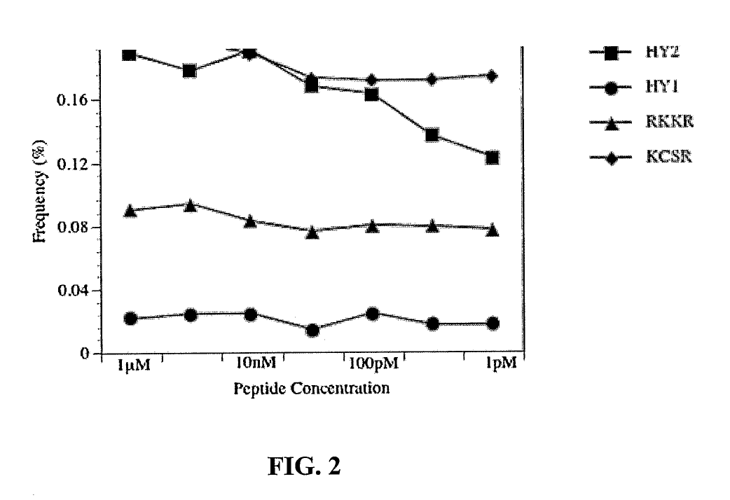

We hypothesized that (1) the formation of precipitates is due to ionic bonds between the positively charged residues in HY2 and the negative charges on CpG molecules and (2) this precipitation enhances stimulation of CTLs. To test these hypotheses, we synthesized two variants of HY1, in which KCSRNR-(SEQ ID NO:1) and RKKRRQ-(SEQ ID NO:3) sequences were added to the amino termini. The KCSRNR sequence (SEQ ID NO:1) was derived from HY2 and the RKKRRQ sequence (SEQ ID NO:3) was derived from the active HIV TAT polypeptide (keeping the length of the CpG-interacting amino acid sequence at six amino acids) (Vives et al., J. Biol. Chem. 272:16010, 1997). The two bipartite polypeptides (but not native HY1) precipitated CpG, supporting our first hypothesis. When mixed and injected with CpG+MPL, the HY1 polypeptide fused to the KCSRNR (SEQ ID NO:1) amino acid sequence strikingly increased the frequency of HY1-specific CTLs (see FIG. 2).

With regard to the (SEQ ID NO:3) RKKRRQ-containing polypeptides, we have also observed that the complete, active HIV TAT polypeptide used in Trojan constructs (Lu et al., J. Immunol. 166:7063, 2001) intensely precipitates CpG to the degree that it is difficult to inject, and it inhibits the initial adjuvant activity of CpG in short-term functional assays. Too many positive charges can block T cell activation and expansion. This is consistent with the observation that poly-Arg (60 residues) can rapidly precipitate CpG (Lingnau et al., Vaccine 20:3498, 2002); such precipitates can be deposited in tissue and have very long life-spans in vivo as evidenced by the ability of these precipitates with associated immunogenic polypeptides to continue priming for at least 372 days (Lingnau et al., Vaccine 20:3498, 2002). The potential for selectively and strongly precipitating both immunogenic polypeptides and CpG provides the means to stimulate long-term and durable CTL responses.

To test the CTL priming cability of the bipartite polypeptides, B6 females were primed with mixtures of CpG adjuvant and HY1, KCSRNR-HY1 (SEQ ID NO:20), RKKRRQ-HY1 (SEQ ID NO:21), and HY2. Spleens were harvested 10 days thereafter for enrichment of CD8⁺ CTLs for primary IFNγ Elispot assays with peptide-pulsed RMA/S cells as stimulators (FIG. 2). Consistent with the results reported in FIGS. 1A and 1B, HY2 primed for greater frequencies of CTLs than HY1 at all tested peptide concentrations. The linkage of the KCSRNR tail with the HY1 target peptide resulted in frequencies of HY1-specific CTLs that were comparable to those obtained with HY2. The RKKRRQ tail (SEQ ID NO:3) increased priming efficiency to an intermediate level compared to HY1 versus KCSRNR-HY1 (SEQ ID NO:1). The HY2, KCSRNR-HY1 (SEQ ID NO:20), and RKKRRQ-HY1 peptides (SEQ ID NO:21) all precipitated CpG oligonucleotides. The increased priming efficiency of KCSRNR-HY1 (SEQ ID NO:20) indicated that the addition of this six amino acid sequence carrying three positively charged residues increased HY1-specific priming. However, the intermediate efficiency associated with the RKKRRQ-HY1 peptide (SEQ ID NO:21) with five positively charged residues suggested that increasing the number of positively charged amino acids was not associated with further increased priming.

### Example 2

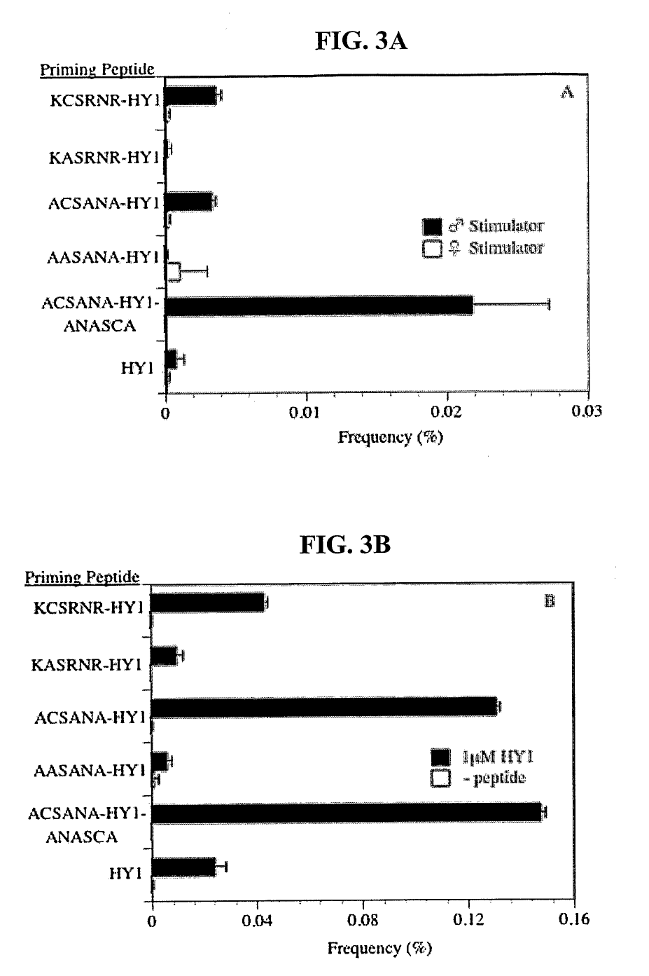

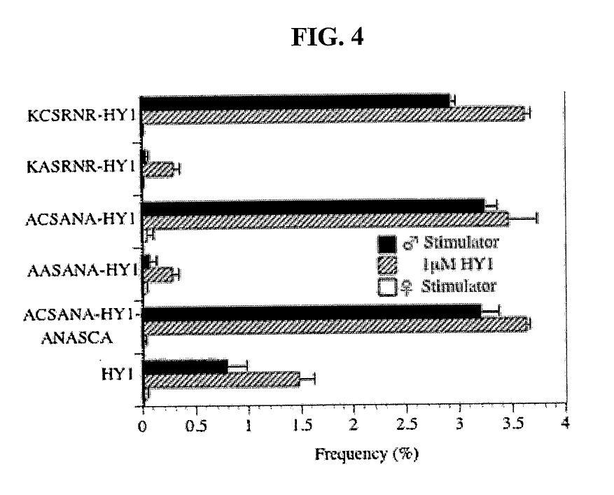

A cysteine residue is important for increased immunogenicity. To examine the role of Arg/Lys residues in the (SEQ ID NO:1) KCSRNR CpG-interacting amino acid sequence of the HY1 bipartite polypeptide, we substituted Ala residues for the three positively charged residues. The altered bipartite polypeptides were each mixed with CpG to prime B6 female mice. Spleen cells were harvested at day 10 and CD8⁺ CTLs were enriched for quantitation in primary and secondary IFNgamma Elispot assays. As in prior experiments, priming with KCSRNR-HY1 (SEQ ID NO:20) resulted in higher frequencies of HY1-specific CTLs in primary Elispot assays than priming with the fully processed HY1 peptide (FIGS. 3A and 3B). This ranking was observed with both syngeneic male (FIG. 3A) and HY1-pulsed syngeneic female spleen cell stimulators (FIG. 3B). To our surprise, substitution of Ala for all Arg/Lys residues in the ACSANA-HY1 (SEQ ID NO:23) polypeptide did not result in the loss of priming efficiency although it did result in the loss of the ability to precipitate CpG. In fact, polypeptide-pulsed female cells stimulated higher frequencies of HY1-specific CTLs from mice primed with ACSANA-HY1 (SEQ ID NO:23) than with KCSRNR-HY1 (SEQ ID NO:20) (FIG. 3B). Confirmatory results were obtained in secondary Elispot assays of CTLs expanded by stimulation with syngeneic male spleen cells in primary MLCs (FIG. 4). Comparable frequencies of HY1-specific CTLs were stimulated with syngeneic male spleen cells and peptide-pulsed spleen cells when responder CTLs were derived from mice primed with KCSRNR-HY1 (SEQ ID NO:20) and ACSANA-HY1 (SEQ ID NO:23).

Synthetic CpG oligonucleotides are typically synthesized with phosphorothioate linkages to reduce susceptibility to nucleases in vivo (Stein et al., Nucl. Acids Res. 16:3209-3221, 1988). One explanation for the pre-eminent role of Cys in driving in vivo priming is the formation of disulfide bonds between the Cys-containing HY1 primotopes and synthetic CpG oligonucleotides. The importance of the Cys residue for in vivo priming with the KCSRNR-HY1 (SEQ ID NO:20) and ACSANA-HY1 (SEQ ID NO:23) primotopes was confirmed by additional Ala substitutions. The KASRNR-HY1 primotope (Cys>Ala) exhibited virtually no priming potential as evaluated by both primary and secondary Elispot assays (FIGS. 3A and 3B, and FIG. 4). However, the KASRNR-HY1 (SEQ ID NO:20) polypeptide still retained the capacity to visibly precipitate CpG. The AASANA-HY1 polypeptide (SEQ ID NO:24) was similar to KASRNR-HY1 in its lack of in vivo priming capacity. These results supported the hypothesis that the single Cys residue was required for optimal HY1-specific priming, potentially through the formation of disulfide bonds with the phosphorothioate linkages of synthetic CpG oligonucleotides. We investigated the possibility that additional Cys residues in the peptides would increase immunogenicity through the ability to cross-link multiple CpG molecules. An HY1 polypeptide was synthesized with amino and carboxy tails carrying single Cys residues: ACSANA-HY1-ANASCA (SEQ ID NO:18). This polypeptide was combined with CpG for priming of B6 females with frequencies of HY1-specific CTLs estimated by primary and secondary Elispot assays. A significant increase in priming efficiency was observed relative to priming with the ACSANA-HY1 (SEQ ID NO:23) polypeptide as revealed by frequencies of HY1-specific CTLs that responded to syngeneic male stimulators in primary Elispot assays (FIG. 3A). Further, comparable frequencies of HY1-specific CTLs were observed in secondary Elispot assays of CD8⁺ CTLs from mice primed with all three polypeptides tailed with Cys residues, i.e., ACSANA (SEQ ID NO:2), KCSRNR (SEQ ID NO:1), and double ACSANA (SEQ ID NO:2) tails (FIG. 4).

### Example 3

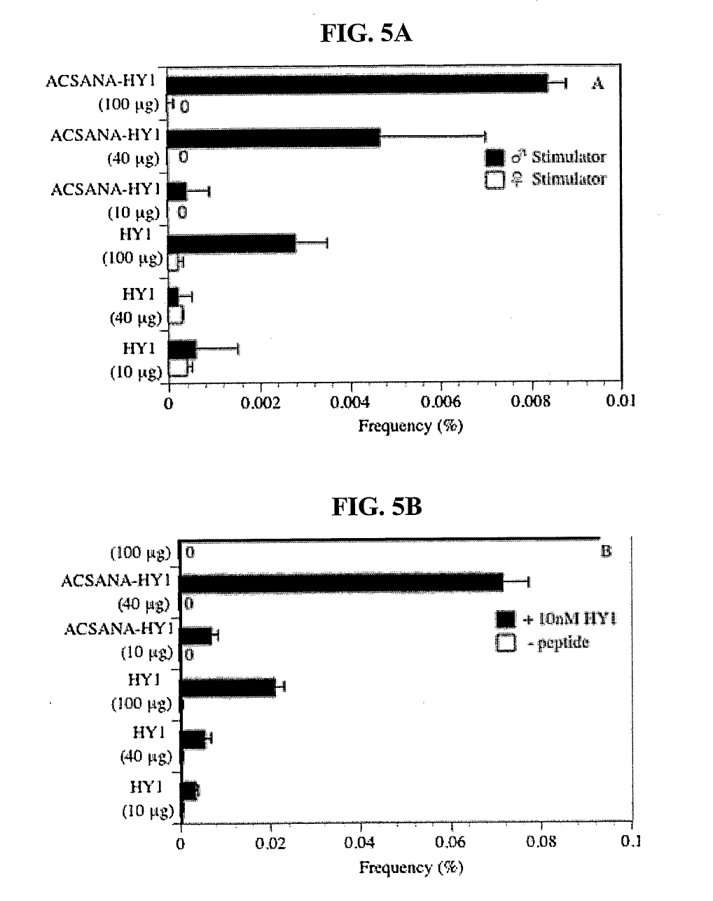

Increased priming efficiency is concentration dependent. We sought to determine if increased priming efficiency with ACSANA-HY1 (SEQ ID NO:23) could still be observed with reduced doses of bipartite polypeptide and CpG. B6 females were primed with mixtures of CpG plus HY1 and ACSANA-HY1 (SEQ ID NO:23) at doses of 100, 40, and 10 μg of each of the two components. Spleens were harvested 10 d later for enrichment of CD8⁺ CTLs for primary and secondary Elispot assays with syngeneic male and female spleen cell stimulators, the latter of which were either untreated or pulsed with HY1. In primary Elispot assays (FIGS. 5A and 5B), priming efficiency was maintained with 40 μg doses of ACSANA-HY1 (SEQ ID NO:23) peptide but not with the fully processed HY1 peptide when tested with syngeneic male stimulators (FIG. 5A) and female stimulators pulsed with 10 nM HY1 peptide (FIG. 5B). No priming was detectable in primary Elispot assays with 10 μg of either ACSANA-HY1 (SEQ ID NO:23) or HY1. Comparable results were obtained in secondary Elispot assays with syngeneic male stimulators and HY1-pulsed syngeneic female stimulators; in the case of HY1-pulsed stimulators, frequencies of CTLs primed with all doses of ACSANA-HY1 (SEQ ID NO:23) were significantly higher than those of CTLs primed with the corresponding doses of HY1.

### Example 4

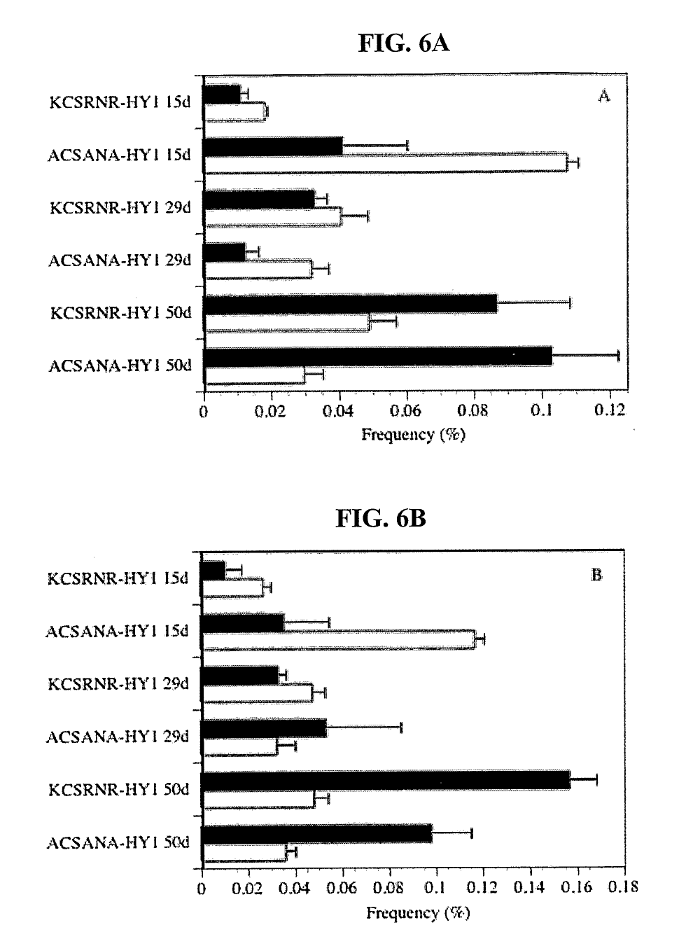

Efficiency of priming by bipartite polypeptides varies over time. We have observed that the presence of Arg/Lys residues in HY1 primotope tails promotes precipitation of CpG, and other investigators have observed that poly-Arg precipitates CpG oligonucleotides that remain deposited at injection sites for extended periods of time with associated extended priming of CTLs (Lingnau et al., Vaccine 20:3498-3508, 2002). We hypothesized that such extended deposition would occur with KCSRNR-HY1 (SEQ ID NO:20) which would then prime for longer periods of time than ACSANA-HY1 (SEQ ID NO:23) that does not precipitate CpG oligonucleotides but strongly primes primary CTL responses. B6 females were primed with CpG plus KCSRNR-HY1 (SEQ ID NO:20) or ACSANA-HY1 (SEQ ID NO:23) and spleens and draining lymph nodes were harvested at 15, 29, and 50 days for primary Elispot assays with peptide-pulsed syngeneic female stimulators (FIGS. 6A and 6B). These results showed that ACSANA-HY1 (SEQ ID NO:23) was most efficient at priming CTLs in both spleen and draining lymph nodes harvested at 15 days with stimulators pulsed with 10 nM (FIG. 6A) and 1 μM HY1 peptide (FIG. 6B). However, this distinction faded at 29 d with KCSRNR-HY1 (SEQ ID NO:20) and ACSANA-HY1 (SEQ ID NO:23) priming for similar frequencies of HY1-specific CTLs in both lymph nodes and spleens. This similarity between ACSANA-HY1 (SEQ ID NO:23) and KCSRNR-HY1 (SEQ ID NO:20) continued through day 50 with comparable levels of priming. Testing at day 50 revealed a shift toward higher frequencies of HY1-specific CTLs in draining lymph nodes than spleens suggesting retention of the two polypeptides at the sites of injection through this timepoint with no consistent difference between ACSANA-HY1 (SEQ ID NO:23) and KCSRNR-HY1 (SEQ ID NO:20).

### Example 5

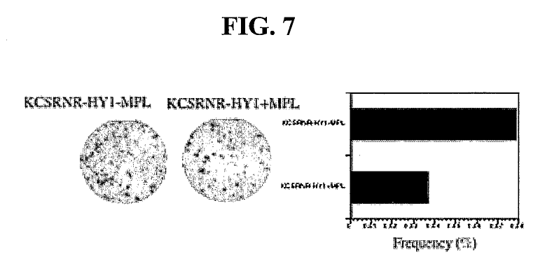

MPL-AF is not required for increased immunogenicity by an immunogenic polypeptide. We investigated the possibility that MPL-AF is dispensable for MiHA polypeptides that are capable of efficiently precipitating CpG. B6 female mice were primed with the KCSRNR-HY1 immunogenic polypeptide+CpG (in our standard concentrations) with or without MPL-AF. The results of the ELISPOT assay for IFNgamma-secreting CTLs demonstrated that the inclusion of MPL-AF in the priming mixtures did not increase the efficiency of stimulation of HY1-specific CTLs and that CpG was sufficient for priming with KCSRNR-HY1 (SEQ ID NO:20) (FIG. 7). In fact, the elimination of MPL-AF appeared to increase the efficiency of priming with only KCSRNR-HY1 (SEQ ID NO:20) and CpG.

### Example 6

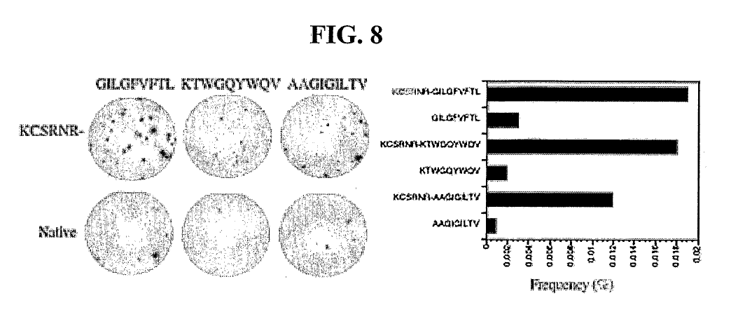

An immunogenic polypeptide increased immunogenicity of melanoma polypeptides. To determine if the addition of KCSRNR (SEQ ID NO:20) amino acid sequence could increase the immunogenicity of polypeptides other than HY1 and H60, a series of HLA-A2-binding polypeptides with KCSRNR (SEQ ID NO:20) amino acid sequences was synthesized. The series included five polypeptides from the tyrosinase, gp100, and MelanA proteins that are specifically expressed by melanocytes and melanoma cells. Since these proteins are normally expressed in mice and humans, tolerance must be broken to achieve priming of CTLs. We also included the immunodominant influenza polypeptide (GILGFVFTL (SEQ ID NO:17)), which is a foreign protein for mice and humans. All of these bipartite polypeptides precipitated CpG whereas none of the native polypeptides precipitated CpG. Recipient mice were HLA-A2 transgenic mice that were selected on the B6 background (Le et al., J. Immunol. 142:1366, 1989). Although these mice expressed HLA-A2 molecules on the cell surface with densities comparable to H2Db molecules, the frequency of influenza-specific CTLs that were restricted by HLA-A2 was drastically reduced in comparison to the frequency of influenza-specific CTLs that were restricted by H2 Db (Le et al., J. Immunol. 142:1366, 1989). Further, HLA-A2-restricted CTL responses to immunogenic polypeptides has been shown to be reduced in these transgenics (Engelhard et al., J. Immunol. 146:1226, 1991) presumably due to the reduced binding of HLA-A2 molecules to mouse b2M and CD8. These mice, obtained from the Jackson Laboratory, had a reduced responsiveness to HLA-A2-restricted polypeptides. HLA-A2 transgenics were primed s.c. with mixtures of 100 μg of CpG mixed with (1) 100 μg of native polypeptides and (2) amounts of (SEQ ID NO:1) KCSRNR-containing polypeptide to yield equimolar CpG:peptide ratios. Spleens were harvested 10 days after immunization and CD8⁺ CTLs were purified by CD8⁺ Negative Selection Kits (Miltenyi Biotec, Auburn, Calif.) for use in primary IFNgamma ELISPOT assays. Stimulators included T2 cells pulsed with titrated concentrations of native melanoma polypeptides. Non-pulsed T2 cells did not stimulate IFNgamma production. The results of this assay demonstrated that the addition of a KCSRNR (SEQ ID NO:1) amino acid sequence resulted in increased priming for the influenza polypeptide and two of the melanoma-specific polypeptides, AAGIGILTV (SEQ ID NO:4) from MelanA and KTWGQYWQV (SEQ ID NO:5) from gp100 (FIG. 8). The responses to (SEQ ID NO:1) KCSRNR-containing polypeptides were ˜6-10 fold higher than the responses to the respective, natural polypeptides. Thus, with no modification to priming technique, we were able to boost the response of recipient mice with handicapped CTL responses to polypeptides presented by HLA-A2 molecules.

### Example 7

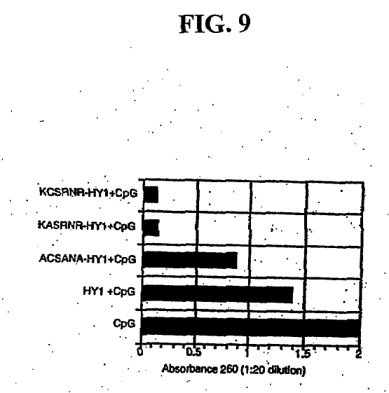

An immunogenic bipartite HY1-Polypeptide precipitated CpG To confirm the presence of CpG in precipitates, HY1 and a series of immunogenic bipartite HY1 polypeptides were mixed with 100 μg CpG at molar ratios of approximately 7:1. After 15 min incubation at room temperature, the mixtures were centrifuged at 1000×g for 15 sec. Optical densities (260/280 nm) of diluted supernatants were measured after centrifugation to estimate the efficiency of precipitation of CpG (FIG. 9). These results confirmed that the KCSRNR-HY1 (SEQ ID NO:20) and KASRNR-HY1 (SEQ ID NO:22) polypeptides precipitated nearly all of the CpG in solution; the natural HY1 polypeptide precipitated ˜30% of the CpG. These results support the hypothesis that the positively charged amino acids are important for precipitation of CpG and indicate that these concentrations of bipartite polypeptide and CpG result in the virtually complete conversion of free CpG to precipitated CpG.

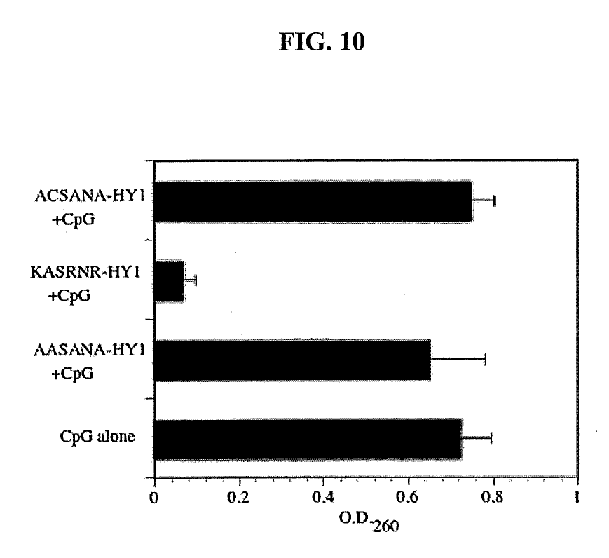

To further investigate the precipitation that occurs when CpG is mixed with HY1-tailed polypeptides, experiments were designed to assess the roles of Arg/Lys and Cys residues in the polypeptide tail. HY1 bipartite polypeptides including KASRNR (SEQ ID NO:11), ACSANA (SEQ ID NO:2), and AASANA (SEQ ID NO:7) tails were used in the analysis. CpG oligonucleotides (6 nmol) were mixed in triplicate with the HY1 bipartite polypeptides (20 nmol) for 15 minutes at room temp. Precipitates were pelleted at 10,000×g, and supernatants were diluted 1/100 for spectrometric analysis at 260 nm. As shown in FIG. 10, KASRNR-HY1 (SEQ ID NO:22) precipitated ≈90% of soluble CpG, and ACSANA-HY1 (SEQ ID NO:23) and AASANA-HY1 (SEQ ID NO:24) had no significant effects on CpG levels. These results support the contention that the initially observed particulate precipitation is the function of interactions between the highly polar nucleic acids and the charged Arg/Lys amino acids of the primotope tails.

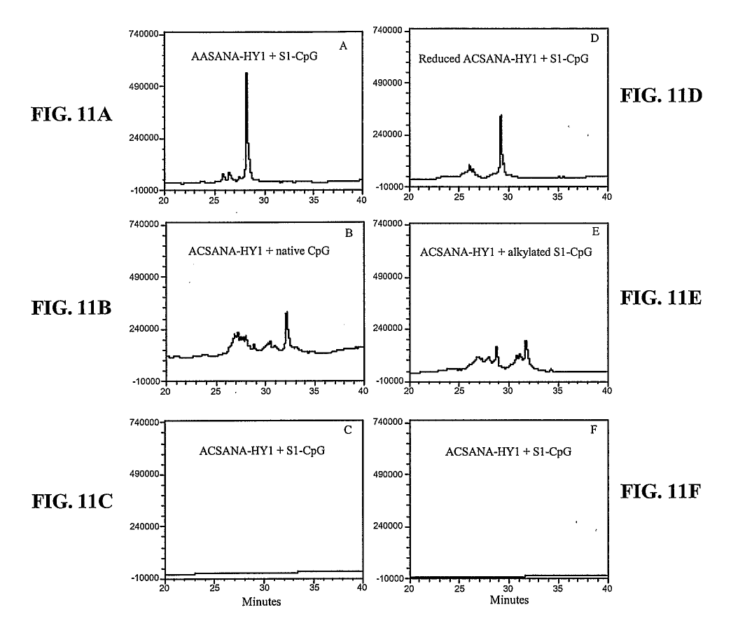

The presence of free peptides in mixtures of peptide plus CpG was quantitated by reverse phase-HPLC, but this method of analysis precluded detection of CpG oligonucleotides and potentially bound peptides. Two forms of CpG oligonucleotide were used in these experiments: (1) native CpG with phosphodiester linkages and (2) CpG with one phosphorothioate linkage between the fourth and fifth nucleotides (S1-CpG). The use of S1-CpG avoided the potential complexity of multiple bond formations. The mixture of AASANA-HY1 (SEQ ID NO:24) with S1-CpG resulted in the detection of the peptide as a single peak by RP-HPLC (FIG. 11A). Likewise, the ACSANA-HY1 (SEQ ID NO:23) signal was detectable following its mixture with native CpG (FIG. 11B) but was undetectable following the mixture of ACSANA-HY1 (SEQ ID NO:23) with S1-CpG (FIG. 11C). The two sets of peaks in the ACSANA-HY1 (SEQ ID NO:23)+native CpG sample were presumed to represent monomers and dimers. These results suggested that the single Cys residue promoted binding of the ACSANA-HY1 (SEQ ID NO:23) bipartite polypeptide to S1-CpG and not native CpG. If this binding were due to disulfide bond formation between Cys-bearing peptides and S1-CpG, then reduction of the peptide:CpG mixture and alkylation of either of the components should eliminate binding. ACSANA-HY1 (SEQ ID NO:23) and S1-CpG were mixed and either analyzed directly by RP-HPLC or reduced with 50 mM dithiothreitol prior to analysis. As shown in FIG. 11D, reduction with dithiothreitol resulted in the release and detection of the ACSANA-HY1 (SEQ ID NO:23) primotope that was absent with the untreated mixture with S1-CpG (FIG. 11F). A single peak eluting as expected for a monomeric peptide was observed under these reducing conditions (FIG. 11D). Further, alkylation of S1-CpG with iodoacetamide prior to admixture to ACSANA-HY1 (SEQ ID NO:23) eliminated binding as indicated by the detection of the putatively monomeric and dimeric ACSANA-HY1 (SEQ ID NO:23) primotopes (FIG. 11E). These results constitute strong evidence that sulfur-bearing CpG oligonucleotides and Cys-bearing polypeptides can form disulfide bonds that can be prevented by alkylation and reduction by dithiothreitol.

### Example 8

Immunogenic polypeptide and CpG increased the efficiency of polypeptide absorption by Langerhans cells. The methods described herein can result in the direct precipitation of CpG by a bipartite, immunogenic, class I-binding polypeptide. The direct participation of both molecules in precipitation ensures that both will be present for the duration of the precipitate and its deposit in vivo. Antigen-presenting cells (APCs) that take up the precipitate also receive CpG molecules for activation and class I-binding polypeptides for presentation to and recognition by CTLs. To test the uptake of the precipitate by APCs, KCSRNR-HY1 (SEQ ID NO:20) and the HY1 polypeptide were stained with Alexa 488, an amine-reactive dye, according to the manufacturer's protocol (Molecular Probes, Eugene, Oreg.). CpG molecules were stained with Texas Red maleimide through the use of a 5′EndTag Nucleic Acid Labeling System that utilizes T4 polynucleotide kinase (Vector Laboratories, Burlingame, Calif.). B10 mice were injected in the ears with a mixture of Alexa 488-polypeptide (10 μg/ear) and Texas Red-CpG (10 ug/ear). Ears were injected with (A) KCSRNR-HY1 (SEQ ID NO:20)+CpG, or (B) HY1+CpG. Ears were harvested at 12 hr, which is a time at which Langerhans cells (LCs) have been shown to have been activated by local CpG injections (Jakob et al., J. Immunol. 161:3042, 1998). Ears were split and LCs extracted by gentle treatment with 0.25% trypsin in Versene. The LC populations were viewed using an Olympus BX51 fluorescent microscope with a triple-pass filter for FITC (Alexa 488) and TRITC (red).

Images of cells revealed that in ears injected with HY1+CpG, LCs absorbed both HY1 polypeptides and CpG molecules within 12 hr, but these molecules appeared to be concentrated within separate compartments in the LCs. In ears injected with KCSRNR-HY1 (SEQ ID NO:20)+CpG, the polypeptides and oligonucleotides were dispersed together throughout some of the LCs. Other LCs displayed concentrated CpG within restricted compartments as well as dispersed polypeptides on the surface of cells. Thus precipitates of CpG and KCSRNR-HY1 (SEQ ID NO:20) can be taken up together by LCs with the subsequently effective and rapid (in comparison to HY1) transport of the polypeptides to the cell surface.

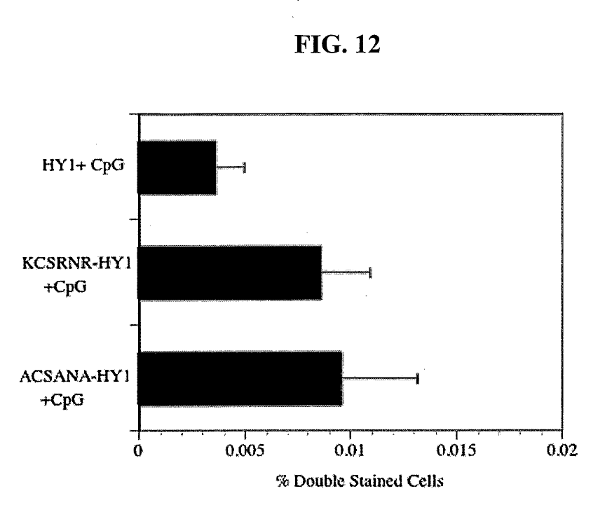

We also investigated the effects of Arg/Lys and Cys residues in tailed HY1 peptides on in vivo migration of Langerhans cells (LCs), which can present class I-bound peptides to CTLs. Activation of dendritic cells (DCs), including LCs, by CpG requires TLR9 expression and results in increased expression of cytokines and co-stimulatory molecules as well as migration to draining lymph nodes. To investigate the effect of the bipartite polypeptides on LC migration, B6 female mice (three per group) were injected in the hind footpads with Texas Red-stained CpG mixed with Alexa 488-conjugated HY1, KCSRNR-HY1 (SEQ ID NO:20), and ACSANA-HY1 (SEQ ID NO:23). Draining popliteal lymph nodes were harvested after 24 hr and lymphoid cells were dissociated to estimate the frequencies of doubly stained cells by fluorescent microscopy. The results presented in FIG. 12 indicate that the addition of KCSRNR (SEQ ID NO:1) and ACSANA (SEQ ID NO:2) tails increased the frequencies of doubly stained cells approximately 2.5-fold relative to the frequencies stimulated by HY1 peptide+CpG.

### Example 9

Bipartite polypeptides increased efficiency of CpG uptake into macrophages. We also investigated the effects of bipartite polypeptides on the mechanisms of uptake of CpG by RAW-264 macrophages. CpG was end-labeled with Texas Red and mixed with the following bipartite polypeptides: AASANA-HY1 (SEQ ID NO:24), ACSANA-HY1 (SEQ ID NO:23), KASRNR-HY1 (SEQ ID NO:22), and KCSRNR-HY1 (SEQ ID NO:20). Texas Red-CpG was mixed with primotopes (5 μg+5 μg) and the mixtures were diluted in growth medium for loading onto RAW cells on coverslips in a POC-R cell cultivation system (LaCon, Staig, Germany) mounted on a LSM 510 laser scanning confocal microscope (Carl Zeiss, Inc., Oberkochen, Germany). The POC-R chamber was heated to 37° C. and the humidification system delivered a 5% CO₂/air mixture. Samples were excited with a 543 nm HeNe laser and viewed through a 63×/1.2 N.A. water C-Apochromat objective. Epi-flourescence was collected through a 560-615 nm band pass filter with the pinhole set to 1 airy unit. Uptake was monitored over five minutes with data acquired at five second intervals. Differential Interface Contrast (DIC) images were collected with the transmitted light detector. Images of 512×512 were collected at 8-bit resolution, and data were analyzed with LSM Image Browser (Carl Zeiss) on a WindowsXP-based PC and with ImageJ (National Institute of Mental Health) on a Macintosh computer.

The bipartite polypeptides were clearly distinguished by their effects on the uptake of CpG. The slowest uptake was observed with (SEQ ID NO:24) AASANA-HY1+CpG; CpG was taken up and distributed throughout the cells. The most rapid uptake of CpG was driven by (SEQ ID NO:22) KASRNR-HY1. Upon addition of (SEQ ID NO:11) KASRNR+CpG, the cells immediately swelled and blebbed followed by the uptake of CpG that continually increased during the 5 min viewing period. Treatment with CpG mixed with (SEQ ID NO:23) ACSANA-HY1 and (SEQ ID NO:20) KCSRNR-HY1 resulted in uptake of CpG that was more rapid than with (SEQ ID NO:24) AASANA-HY1 and was concentrated in discrete cellular locations. This focused uptake was followed by pronounced movement of the cells that was only observed with these Cys-bearing primotopes+CpG. It was clear that the addition of Cys in (SEQ ID NO:20) KCSRNR-HY1 had blunted the rapid and apparently deleterious effects of the positively charged primotope. Importantly, the focused uptake of CpG observed with Cys-bearing primotopes correlated with the capacity to drive the increased CTL expansion.

### Example 10

Known antigens have features of immunogenic polypeptides. The HY2 polypeptide, KCSRNRQYL (SEQ ID NO:10), includes amino acids that are recognized by CTLs, control binding to H2 Db molecules, mediate binding to CpG (Cys), and precipitate CpG (Arg and Lys). The combination of these characteristics can contribute to the relatively high immunogenicity of HY2 when administered with CpG. Further, the use of the first six amino acids (KCSRNR) (SEQ ID NO:1) of a bipartite HY1 and other polypeptides increases their immunogenicity when combined with CpG. These observations led us to examine the amino acid sequences of proteins that include a variety of immunogenic polypeptides and search for the presence of “natural immunogenic polypeptides” that include not only the recognition polypeptide but also flanking sequences that include Cys and positively charged residues that can bind to CpG. A precursory examination of known tumor, viral, and minor histocompatibility antigens revealed a remarkable tendency for class I-binding polypeptides to be flanked by multiple Arg and/or Lys residues. Table 1 illustrates such linkage in segments of three tumor-related proteins and one viral-encoded protein (class I-binding polypeptides in bold).

TABLE 1

Known antigens are flanked by Arg and/or
Lys residues.

| | |
|---|---|
| Gene | Sequence |

| | |
|---|---|
| HER-2/neu | IVSAVVGILLVVVLGVVFGILIKRRQQKIRKYTMRRL |
| | LQETELV* |
| | (SEQ ID NO: 12) |
| MelanA | AAGIGILTVILGVLLLIGCWYCRRRNGYR |
| | (SEQ ID NO: 13) |
| PSA-1 | FLTPKKLQCVDLHVISNDVCAQVHPQKVTKFMLCAGRW |
| | TGGK |
| | (SEQ ID NO: 14) |
| Adenovirus | LIVIGILILSVILYFIFCRQIPNVHRNSKRR |
| 3 (E3) | (SEQ ID NO: 15) |

*Residues in bold are class I-binding polypeptides; Arg, Cys, and Lys residues are underlined

Arg is selectively and sparsely used in proteins (Dyer, J. Biol. Education 5:15, 1971; King and Jukes, Science 164:788, 1969), an indication that the presence of Arg residues in flanking regions of class I-binding polypeptides may not be a random occurrence. The observed regions of positively-charged amino acids are either a part of the polypeptides themselves or tend to be located up to 40 amino acids away from the polypeptide. We observed an apparent correlation between these positively charged segments that carry immunogenic polypeptides and genes whose functions depend on the functions of Arg residues. Proteins that functionally depend on Arg residues, such as DNA binding proteins that must cross nuclear membranes and bind to DNA and members of the hydrophobic ABC transporter family, are highly represented as antigen sources. Continued analysis of known antigens further revealed that multiple immunogenic polypeptides can be found in clusters that share a linkage with a region of positively-charged amino acids. Thus, the membrane transport of one relatively large region has the potential to deliver more than one epitope simultaneously.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. A composition comprising a polypeptide and a CpG molecule, wherein said polypeptide comprises a cytotoxic T lymphocyte-activating amino acid sequence and a CpG-interacting amino acid sequence, wherein said cytotoxic T lymphocyte-activating amino acid sequence is heterologous to said CpG-interacting amino acid sequence, wherein said CpG-interacting amino acid sequence comprises at least one cysteine residue, and wherein said CpG molecule comprises at least one sulfur atom.

2. The composition of claim 1, wherein said CpG-interacting amino acid sequence further comprises at least one positively charged amino acid.

3. The composition of claim 1, wherein said CpG-interacting amino acid sequence comprises no more than 15 amino acid residues.

4-5. (canceled)

6. The composition of claim 1, wherein said CpG-interacting amino acid sequence comprises a B-X, X-B, or B-X-B sequence, wherein B is a positively charged amino acid residue and X is an amino acid residue.

7. The composition of claim 1, wherein said CpG-interacting amino acid sequence comprises an B-X-B-X-B sequence, wherein B is a positively charged amino acid residue and X is an amino acid residue.

8. The composition of claim 1, wherein said CpG-interacting amino acid sequence comprises at least two cysteine residues.

9. The composition of claim 1, wherein said CpG-interacting amino acid sequence comprises at least 4 positively charged amino acid residues.

10. The composition of claim 1, wherein at least one of said at least one cysteine residue of said CpG-interacting amino acid sequence is adjacent to a positively charged amino acid residue.

11. The composition of claim 10, wherein said CpG-interacting amino acid sequence comprises the sequence set forth in SEQ ID NO:1 (KCSRNR).

12. The composition of claim 1, wherein said CpG-interacting amino acid sequence consists essentially of the sequence set forth in SEQ ID NO:1 (KCSRNR).

13. The composition of claim 1, wherein said CpG-interacting amino acid sequence consists essentially of the sequence set forth in SEQ ID NO:2 (ACSANA).

14. The composition of claim 13, wherein said at least one positively charged amino acid residue is an arginine.

15. The composition of claim 13, wherein said at least one positively charged amino acid residue is a lysine.

16. The composition of claim 1, wherein said cytotoxic T lymphocyte-activating amino acid sequence comprises no more than 50 amino acid residues.

17-19. (canceled)

20. The composition of claim 1, wherein said polypeptide is less than 50 amino acid residues in length.

21-23. (canceled)

24. The composition of claim 1, wherein said CpG molecule comprises a phosphorothioate linkage.

25. (canceled)

26. A method for producing a composition having enhanced immunogenicity, said method comprising:
(a) obtaining a polypeptide having a cytotoxic T lymphocyte-activating amino acid sequence and a CpG-interacting amino acid sequence, wherein said cytotoxic T lymphocyte-activating amino acid sequence is heterologous to said CpG-interacting amino acid sequence, and wherein said CpG-interacting amino acid sequence comprises at least one cysteine residue; and
(b) contacting said polypeptide to a CpG molecule comprising a sulfur atom to form said composition.

27. The method of claim 26, wherein said CpG-interacting amino acid sequence further comprises at least one positively charged amino acid.

28-30. (canceled)

31. A method for activating a cytotoxic T lymphocyte within a mammal, said method comprising administering a composition comprising a polypeptide and a CpG molecule to said mammal, wherein said polypeptide comprises a cytotoxic T lymphocyte-activating amino acid sequence and a CpG-interacting amino acid sequence, wherein said cytotoxic T lymphocyte-activating amino acid sequence is heterologous to said CpG-interacting amino acid sequence, wherein said CpG-interacting amino acid sequence comprises at least one cysteine residue, and wherein said CpG molecule comprises a sulfur atom.

32. The method of claim 31, wherein said CpG-interacting amino acid sequence further comprises at least one positively charged amino acid.

33-37. (canceled)


SEQUENCE LISTING (24 sequences)

SEQ ID NO: 1 - protein, 6 aa, Artificial Sequence
  Synthetically generated peptide
KCSRNR

SEQ ID NO: 2 - protein, 6 aa, Artificial Sequence
  Synthetically generated peptide
ACSANA

SEQ ID NO: 3 - protein, 6 aa, Artificial Sequence
  Synthetically generated peptide
RKKRRQ

SEQ ID NO: 4 - protein, 9 aa, Homo sapiens
AAGIGILTV

SEQ ID NO: 5 - protein, 8 aa, Homo sapiens
KTWQYWQV

SEQ ID NO: 6 - protein, 8 aa, Influenza virus
GILGFVFT

SEQ ID NO: 7 - protein, 6 aa, Artificial Sequence
  Synthetically generated peptide
AASANA

SEQ ID NO: 8 - DNA, 6 bp, Artificial Sequence
  Consensus motif
rrcgyy

SEQ ID NO: 9 - protein, 9 aa, Artificial Sequence
  Synthetically generated peptide
WMHHNMDLI

SEQ ID NO: 10 - protein, 9 aa, Artificial Sequence
  Synthetically generated peptide
KCSRNRQYL

SEQ ID NO: 11 - protein, 6 aa, Artificial Sequence
  Synthetically generated peptide
KASRNR

SEQ ID NO: 12 - protein, 44 aa, Homo sapiens
IVSAVVGILLVVVLGVVFGILIKRRQQKIRKYTMRRLLQETELV

SEQ ID NO: 13 - protein, 29 aa, Homo sapiens
AAGIGILTVILGVLLLIGCWYCRRRNGYR

SEQ ID NO: 14 - protein, 42 aa, Homo sapiens
FLTPKKLQCVDLHVISNDVCAQVHPQKVTKFMLCAGRWTGGK

SEQ ID NO: 15 - protein, 31 aa, Adenovirus
LIVIGILILSVILYFIFCRQIPNVHRNSKRR

SEQ ID NO: 16 - DNA, 20 bp, Artificial Sequence
  Primer
tccatgacgttcctgacgtt

SEQ ID NO: 17 - protein, 9 aa, Influenza virus
GILGFVFTL

SEQ ID NO: 18 - protein, 21 aa, Artificial Sequence
  Synthetically generated peptide
ACSANAWMHHNMDLIANASCA

SEQ ID NO: 19 - protein, 8 aa, Artificial Sequence
  Synthetically generated peptide
LTFNYRNL

SEQ ID NO: 20 - protein, 15 aa, Artificial Sequence
  Synthetically generated peptide
KCSRNRWMHHNMDLI

SEQ ID NO: 21 - protein, 15 aa, Artificial Sequence
  Synthetically generated peptide
RKKRRQWMHHNMDLI

SEQ ID NO: 22 - protein, 15 aa, Artificial Sequence
  Synthetically generated peptide
KASRNRWMHHNMDLI

SEQ ID NO: 23 - protein, 15 aa, Artificial Sequence
  Synthetically generated peptide
ACSANAWMHHNMDLI

SEQ ID NO: 24 - protein, 15 aa, Artificial Sequence
  Synthetically generated peptide
AASANAWMHHNMDLI